(19) United States
(12) Patent Application Publication
(10) Pub. No.: US 2014/0004528 A1
(43) Pub. Date: Jan. 2, 2014

(54) Spectroscopic Troponin I Detection and Quantification Using Plasmonic Nano-Materials

(71) Applicant: Nanosensing Technologies, Inc., Panama City, PA (US)

(72) Inventors: Patricia Maria Albuquerque de Farias, Recife (BR); Arnaldo Cesar Dantas dos Santos Andrade, Recife (BR); Josivandro do Nascimento Silva, Recife (BR); Jamil Saade, Recife (BR)

(73) Assignee: NANOSENSING TECHNOLOGIES, INC., Panama City (PA)

(21) Appl. No.: 13/706,901

(22) Filed: Dec. 6, 2012

Related U.S. Application Data
(60) Provisional application No. 61/568,768, filed on Dec. 9, 2011.
(60) Provisional application No. 61/568,789, filed on Dec. 9, 2011.

(51) Int. Cl. G01N 33/68

(52) U.S. Cl. CPC G01N 33/6887
USPC 435/7.1; 436/501; 435/287.2; 422/69

(57) ABSTRACT

A system, and its associated methodology, for detecting cTnl includes a patient sample such as whole blood, serum or plasma, anti-cTnl antibodies conjugated with ligand-capped metallic nanoparticles, and a spectrophotometer operable to detect a shift of wavelength in light resulting from binding of cTnl to the anti-cTnl antibodies conjugated with ligand-capped metallic nanoparticles. By measuring changes in Localized Surface Plasmon Resonance of the anti-cTnl antibodies conjugated with ligand-capped metallic nanoparticles the presence of cTnl can be detected.

### RELATED APPLICATION

The present application relates to and claims the benefit of priority to U.S. Provisional Patent Applications Nos. 61/568,768 and 61/568,789 both filed Dec. 9, 2011, which are hereby incorporated by reference in their entirety for all purposes as if fully set forth herein.

### BACKGROUND OF THE INVENTION

1. Field of the Invention

Embodiments of the present invention relate, in general, to Troponin I detection and, more particularly, to a methodology for cardiac-specific Troponin I detection and quantification using plasmonic nano-materials.

2. Relevant Background

Heart attacks (myocardial infarctions) are major causes of death and disability. The accurate and rapid diagnosis of myocardial infarction is important for both efficiently treating the patient and minimizing the costs to the health care system that are incurred by identifying individuals who do not need treatment.

Electrocardiography (ECG) and measurement of cardiac troponins are the current diagnostic cornerstones and complement the clinical assessment. ECG by itself is often insufficient to diagnose an acute coronary syndrome or acute myocardial infarction. However, cardiac troponins, which are structural proteins unique to the heart, are sensitive and specific biochemical markers of myocardial damage.

Cardiac-specific Troponin I (cTnl) is a protein subunit of the cardiac troponin complex. During the myocardial damage process, the troponin complex dissociates and the individual protein components, including cTnl, cardiac-specific Troponin T (cTnT) and cardiac-specific Troponin C (cTnC), are released into the bloodstream. Due to its high specificity and sensitivity for myocardial cell injury, cardiac-specific Troponin I is the “gold standard” biomaker for the clinical diagnosis of acute myocardial damage. In addition to the rapid rise of cTnl concentrations in the blood after the onset of acute myocardial injuries, cTnl concentrations remain elevated for up to 4-10 days after the cardiac event. Additionally, the blood concentrations of cTnl correlate with the degree of heart damage.

Early stage detection methods that can quickly measure very low concentrations of cTnl with high sensitivity are needed for the early diagnosis of Acute Myocardial Infarction (AMI) and progress management of patients. Current cTnl detection methods are based on traditional enzyme linked immunosorbent assay (ELISA) methods which capture anti-cTnl antibodies against cTnl that are firstly immobilized onto the surface of a plastic well, followed by adding a patient sample containing cTnl for binding to the anti-cTnl antibody. Then an enzyme labeled detector anti-cTnl antibody is allowed to bind with the immobilized cTnl. In each step mentioned above, numerous wash cycles are necessary in order to remove the unbound antibody or antigen from the well. Eventually, enzymatic substrate is added and cTnl is detected by an enzyme-dependent color change reaction. The entire ELISA process can take several hours to days to accomplish, which is labor-intensive and time consuming. Early detection and treatment of AMI substantially improves a patient's prognosis. A need therefore exists for a reliable, quick test to determine the presence of cTnl and thus the occurrence of an AMI.

One promising means of biological and chemical sensing is plasmonics. Plasmonics is the study of light-matter interactions in which materials that possess a negative real and small positive imaginary dielectric constant are capable of supporting surface plasmon resonance (SPR). This resonance is a coherent oscillation of the surface conduction electrons excited by electromagnetic (EM) radiation.

Propagated surface plasmon resonance (PSPR) relates to a phenomenon when an incidence light emitted from a light source reaches the surface of a metal film at a fixed incident angle. In such an instance the light intensity reflected from a surface of the metal film picked up by a photo detector is approaching zero, i.e., the reflectance of the metal film is approaching zero while the light beam not reflected propagates at a given speed in a direction along the interface and excites the plasmon on the surface of the metal film to resonate. However, light in a sample medium cannot naturally excite PSPR and a high refractive index prism or grating is required for coupling.

By comparison, Localized Surface Plasmon Resonance (LSPR) is defined as collective charge density oscillations restricted in the neighborhood of nanoparticles excited by an electromagnetic field with a specific frequency. LSPR may be set without utilizing the prism or grating for light coupling. LSPR is a possible excited state of the metallic nanoparticle electron system, which can be excited by photons or, equivalent, by an electromagnetic field of light incident on the particle.

LSPR excitation is a consequence of the inter-electronic (collective) interactions of the electrons combined with spatial confinement of the conduction band electron system within a conductive nanoparticle volume. An electron density wave is formed with a frequency/wavelength/energy that depends on the electronic structure of the nanoparticle, its geometry, size and dielectric environment. The spectral sensitivity of the LSPR, that is the amount of spectral shift of the LSPR along the wavelength/frequency/energy axis (alternatively, a spectral sensitivity of a LSPR) is measurable to events taking place on the surface of and close to the nanoparticles). This characteristic creates the possibility to use “plasmonic” nanoparticles as transducers in sensors. Most of these reported applications of LSPR in plasmonic biosensors rely on the sensitivity of the nanoparticle LSPR to the dielectric constant of the surrounding medium, opening up a route to “refractive index sensing” where adsorbate-induced changes in the local dielectric environment are utilized for detection of e.g. molecular binding events on the nanoparticle surface and in the particle nano-environment.

Very small sensors can be made by using LSPR technique with a simple optical set up. Similar to conventional PSPR, the resonance condition may detect an immediate change in the interfacial refractive index (R1) of the surrounding medium as well as the bio-molecular interactions at the colloid-solution interface.

In general, label-free sensing using metallic nanoparticles can be achieved by analyte-mediated metallic nanoparticle aggregation. This concept is that the surface of the metallic nanoparticles is modified with recognition molecules that specifically bind the analyte of interest. The metallic nanoparticles modified with recognition molecules are spatially brought very close to each other when the recognition molecule binds to the target molecule after adding the sample containing the analyte, which initiates the coupling of plasmon resonances from adjacent metallic nanoparticles. As a result, the surface plasmon resonance bands of aggregated metallic nanoparticles will be broadened and red-shifted as a function of the amount of analyte. These changes can be detected on a conventional UV-visible spectrophotometer.

LSPR offers a platform for biological and chemical sensing. Bioassays that utilize this optical phenomenon include but are not limited to nucleic acid hybridization assays, protein detection assays, surface-enhanced Raman spectroscopy (SERS) Raman labels, intracellular detection and metal enhanced fluorescence (MEF).

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### SUMMARY OF THE INVENTION

Disclosed hereafter by way of example are systems and their associated methodologies for detecting cardiac-specific Troponin I (cTnl) using polystyrene sulfonate capped metallic nanoparticles conjugated with anti-cTnl antibodies. According to one embodiment of the present invention cTnl is detected by dispersing metallic nanoparticles in an aqueous Cetyl Trimethyl Ammonium Bromide (CTAB) solution forming a plurality of dispersed metallic nanoparticles. These metallic nanoparticles are then coated with a ligand forming a plurality of ligand-capped metallic nanoparticles. In one embodiment of the present invention the ligand is polystyrene sulfonate (PSS) resulting in the formation of a plurality of PSS-capped metallic nanoparticles. A dispersion containing the plurality of PSS-capped metallic nanoparticles is maintained at a pH of substantially 7.0. The process continues by conjugating the plurality of PSS-capped metallic nanoparticles with anti-Troponin I (anti-cTnl) antibodies. These anti-cTnl antibodies conjugated with PSS-capped metallic nanoparticles are thereafter mixed with a patient sample wherein the cTnl within the sample binds with the anti-cTnl antibodies conjugated with PSS-capped metallic nanoparticles. Again a dispersion containing the plurality of anti-cTnl antibodies conjugated with PSS-capped metallic nanoparticles has a pH of substantially 7.0. Lastly a presence of cTnl bound to the anti-Tnl antibodies conjugated with PSS-capped metallic nanoparticles is detected using a signal generated by Localized Surface Plasmon Resonance (LSPR).

An additional feature of the present invention includes that the anti-cTnl antibodies conjugated with ligand-capped metallic nanoparticles bound with cTnl shifts the signal generated by Localized Surface Plasmon Resonance relative to the signal generated by unbound anti-Tnl antibodies conjugated with ligand-capped metallic nanoparticles. Moreover the presence of cTnl bound to the anti-Tnl antibodies conjugated with ligand-capped metallic nanoparticles can be detected at concentrations of cTnl from 0.002 ng/mL to and including 40 ng/mL.

Another feature of the present invention is that the plurality of ligand-capped metallic nanoparticles and the plurality of anti-cTnl antibodies conjugated with ligand-capped metallic nanoparticles are dispersed in a Phosphate Buffered Saline (PBS) buffer. Furthermore the dispersion is, according to one embodiment, void of acidic material and specifically hydrochloric acid (HCl).

The metallic nanoparticles of the present invention are, in one embodiment, nanorods and more specifically gold nanorods. These gold nanorods can have an aspect ratio from 4.0 to 5.0.

The detection of cTnl, according to one embodiment of the present invention, can occur in a sample of whole blood or, in other embodiments, serum or plasma.

According to another embodiment of the present invention a system for detecting cTnl includes one or more patient samples such as whole blood, serum or plasma, anti-cTnl antibodies conjugated with ligand-capped metallic nanoparticles, and a spectrophotometer operable to detect a shift in wavelength of light resulting from binding of cTnl to the anti-cTnl antibodies conjugated with ligand-capped metallic nanoparticles. In one embodiment of the present invention the spectrophotometer utilizes changes in Localized Surface Plasmon Resonance of the anti-cTnl antibodies conjugated with ligand-capped metallic nanoparticles to detect the presence of cTnl. These and other feature of the present invention are further described in detail below.

The features and advantages described in this disclosure and in the following detailed description are not all-inclusive. Many additional features and advantages will be apparent to one of ordinary skill in the relevant art in view of the drawings, specification, and claims hereof. Moreover, it should be noted that the language used in the specification has been principally selected for readability and instructional purposes and may not have been selected to delineate or circumscribe the inventive subject matter; reference to the claims is necessary to determine such inventive subject matter.

### BRIEF DESCRIPTION OF THE DRAWINGS

The aforementioned and other features and objects of the present invention and the manner of attaining them will become more apparent, and the invention itself will be best understood, by reference to the following description of one or more embodiments taken in conjunction with the accompanying drawings, wherein:

FIG. 1 shows a schematic illustration of modified metallic nanoparticles interaction with anti-cTnl;

FIG. 2 shows absorption spectrum obtained for gold non-functional and for functional (MNPs/Anti-cTnl) nanorods; and

FIG. 3 shows absorption spectra obtained for distinct cTnl concentrations in blood samples.

FIG. 4 is a flowchart of one method embodiment according to the present invention for detection of cardiac cTnl using conjugated PSS capped metallic nanoparticles.

FIG. 5 shows a high-level block diagram representation or a point of care device according to one embodiment of the present invention; and

FIG. 6 presents a high level block diagram showing the functional relationship between components of a point of care device for cardiac injury detection according to one embodiment of the present invention.

The Figures depict embodiments of the present invention for purposes of illustration only. One skilled in the art will readily recognize from the following discussion that alternative embodiments of the structures and methods illustrated herein may be employed without departing from the principles of the invention described herein.

### DESCRIPTION OF THE INVENTION

Embodiments of the present invention are hereafter described in detail with reference to the accompanying Figures. Although the invention has been described and illustrated with a certain degree of particularity, it is understood that the present disclosure has been made only by way of example and that numerous changes in the combination and arrangement of parts can be resorted to by those skilled in the art without departing from the spirit and scope of the invention.

The following description with reference to the accompanying drawings is provided to assist in a comprehensive understanding of exemplary embodiments of the present invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. Also, descriptions of well-known functions and constructions are omitted for clarity and conciseness.

Unless defined herein, the terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Like numbers refer to like elements throughout. In the figures, the sizes of certain lines, layers, components, elements or features may be exaggerated for clarity. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

An “antibody,” also known as an immunoglobulin (Ig), is a large Y-shaped protein produced by B-cells that is used by the immune system to identify and neutralize foreign objects such as bacteria and viruses. The antibody recognizes a unique part of the foreign target, called an antigen. Each tip of the “Y” of an antibody contains a paratope (a structure analogous to a lock) that is specific for one particular epitope (similarly analogous to a key) on an antigen, allowing these two structures to bind together with precision. Using this binding mechanism, an antibody can tag a microbe or an infected cell for attack by other parts of the immune system, or can neutralize its target directly (for example, by blocking a part of a microbe that is essential for its invasion and survival).

An “antigen” is a substance that evokes the production of one or more antibodies. Each antibody binds to a specific antigen by way of an interaction similar to the fit between a lock and a key. The substance may be from the external environment or formed within the body.

The term “nanoparticle” is defined as a small object that behaves as a whole unit with respect to its transport and properties. Coarse particles cover a range between 10,000 and 2,500 nanometers. Fine particles are sized between 2,500 and 100 nanometers. Ultra-fine particles, or nanoparticles, are sized between 100 and 1 nanometers.

A “nanorod” is a morphology of nanoparticles in which each dimension ranges from 1-100 nm. They may be synthesized from metals or semiconducting materials. Standard aspect ratios (length divided by width) are 3-5. Nanorods are typically produced by direct chemical synthesis.

An “epitope,” also known as antigenic determinant, is the part of an antigen that is recognized by the immune system, specifically by antibodies, B cells, or T cells. The part of an antibody that recognizes the epitope is called a paratope. Although epitopes are usually non-self proteins, sequences derived from the host that can be recognized are also epitopes.

An “immunoassay” is a specific type of biochemical test that measures the presence or concentration of a substance (referred to as the “analyte”) in solutions that frequently contain a complex mixture of substances.

The term “analyte” as used herein refers to a compound or composition being analyzed in a liquid sample. The samples which are usable in the present invention may be selected from any samples containing such an analyte. Examples include physiological fluid such as urine, serum, plasma, blood, saliva, spinal fluid, ocular liquid, amniotic fluid, etc., food such as milk and wine, chemical treatment stream such as domestic waste water. Analytes that can be examined in the present invention are largely classified into a complete antigen and a hapten (incomplete antigen). The complete antigen refers to an antigenic substance which itself has the ability to induce antibody production (immunogenicity), and mainly includes peptide hormones having high molecular weights. The hapten (incomplete antigen) refers to a material which can bind to an antibody but has no ability to induce antibody production by itself, and includes peptides having relatively low molecular weights (molecular weights of about 1,000 or less). Haptens acquire the ability to induce antibody production when bound to a protein such as bovine serum albumin.

The term “sensitivity” as used herein refers to a minimum quantity of a conjugate of a captor, detector and analyte which can be detected.

The cardiac isotype of the myofibrillar contractile protein, Troponin I, is uniquely located in cardiac muscle. Tnl is the inhibitory subunit of Troponin, a thin filament regulatory protein complex, which confers calcium sensitivity to the cardiac and striated muscle. Troponin I exists in three isoforms: two skeletal Tnl (fast and slow) isoforms (Molecular Weight: 19,800 daltons) and a cardiac Tnl isoform with an additional 31 residues (human Tnl) on the N-terminus resulting in a molecular weight of 23,000 daltons.

Cardiac Tnl is found in human serum rapidly following a myocardial infarction. It reaches a peak level after approximately 18 to 24 hours and can remain at elevated levels in the blood stream for up to 6 to 7 days. Thus, immunoassays which can test for human cTnl are valuable to the medical community and to the public.

It will be understood that when an element is referred to as being “on,” “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of a device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal,” and the like are used herein for the purpose of explanation only unless specifically indicated otherwise.

Also included in the description are flowcharts depicting examples of the methodology which may be used to detect the presence of cTnl in a patient blood sample. The blocks of the flowchart illustrations support combinations of means for performing the specified functions and combinations of steps for performing the specified functions. It will also be understood that each block of the flowchart illustrations, and combinations of blocks in the flowchart illustrations, can be implemented by special purpose hardware including hardware-based computer systems that perform the specified functions or steps, or combinations of special purpose hardware and computer instructions.

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise. Thus, for example, reference to “a component surface” includes reference to one or more of such surfaces.

Disclosed hereafter by way of example are one or more methodologies for detecting and quantifying cardiac-specific Troponin I (cTnl) in body fluids. The presence of cTnl in the blood, above a nominal concentration, is diagnostic for damaged heart muscle. Up to 80% of patients with acute myocardial infarction (AMI) will have an elevated cTnl level within 2-3 hours of emergency department arrival, versus 6-9 hours or more with creatine kinase and/or myoglobin and other cardiac markers. According to one embodiment of the present invention, cTnl is detected and quantified using conductive nanoparticles. Unlike the tests of the prior art that can take hours or even days to complete, this test can render results in 4 minutes or less at significantly lower costs and detect cTnl concentrations in the range: 0.002 ng/ml cTnl 40 ng/ml.

The detection of cTnl, according to one embodiment of the present invention, is accomplished by binding to cTnl to anti-cTnl antibodies. These anti-cTnl antibodies are conjugated with metallic nanoparticles, specifically ligand-capped metallic nanoparticles. The presence of metallic nanoparticles can be detected by spectrometry. Moreover, the dielectric environment of the nanoparticles conjugated to the anti-cTnl antibodies changes upon its binding to cTnl. The present invention identifies and measures this change to confirm not only the presence of cTnl but also its concentration.

According to one embodiment of the present invention, cTnl is detected with an antibody specific for this protein. As previously described, an antibody is a large Y-shaped protein produced by B-cells. Each tip of the “Y” of an antibody contains a paratope (a structure analogous to a lock) that is specific for one particular epitope (similarly analogous to a key) on an antigen (in this case, cTnl), allowing these two proteins to bind together with precision. Because this antibody can only specifically bind to cTnl, it is termed an anti-cTnl antibody.

cTnl exists in multiple forms. As cTnl is a component of the Troponin complex responsible for the regulation of muscle contraction, it interacts with the other components of the complex, namely cardiac-specific Troponin T (cTnT) and cardiac-specific Troponin C (cTnC). For example, in the presence of intracellular calcium, there is a strong interaction between cTnl and cTnC. This binary complex is the major form of cTnl in the blood of patients with acute myocardial infarction (AMI) and only a small fraction of cTnl is in the free form. Thus, all forms of cTnl should be considered when designing methodologies directed toward cTnl detection. Accordingly, in one embodiment of the present invention, the anti-cTnl antibodies specifically bind free cTnl. In another embodiment of the present invention, the anti-cTnl antibodies specifically bind complexed cTnl, wherein the complex is a binary complex comprising one other Troponin component selected from the group consisting of cTnC and cTnT. In yet another embodiment of the present invention, the anti-cTnl antibodies specifically bind complexed cTnl, wherein the complex is a ternary complex comprising two other Troponin components selected from the group consisting of cTnC and cTnT. In yet another embodiment of the present invention, the anti-cTnl antibodies specifically bind both free and complexed cTnl.

The N- and C-terminal ends of cTnl are susceptible to proteolytic degradation both in vivo and after sampling. Therefore, in one embodiment of the present invention, the anti-cTnl antibodies are against specific epitopes in the central part (located approximately between amino acid residues 28 and 110) of cTnl.

After an antibody (in this case, anti-cTnl antibody) binds to an antigen (in this case, cTnl), a means must be used to either directly or indirectly detect the antibody-antigen complex. Unlike detection systems of the prior art which rely on immunofluorescence or chemiluminescence, one or more embodiments of the present invention detect the antibody-antigen complex via a signal generated by Localized Surface Plasmon Resonance (LSPR).

LSPR is an optical phenomena that results from light interacting with conductive nanoparticles. By definition, a nanoparticle is a microscopic particle whose size is measured in nanometers. According to one embodiment of the present invention, the antibodies (in this case, anti-cTnl antibodies) are conjugated with conductive nanoparticles comprised of metal. These nanoparticles are biocompatible and their surfaces can be easily modified. These as well as other attributes make metallic nanoparticles superbly suited for a myriad of bioassays and as nanoscale platforms for biosensing and qualitative/quantitative tests and diagnostics assays.

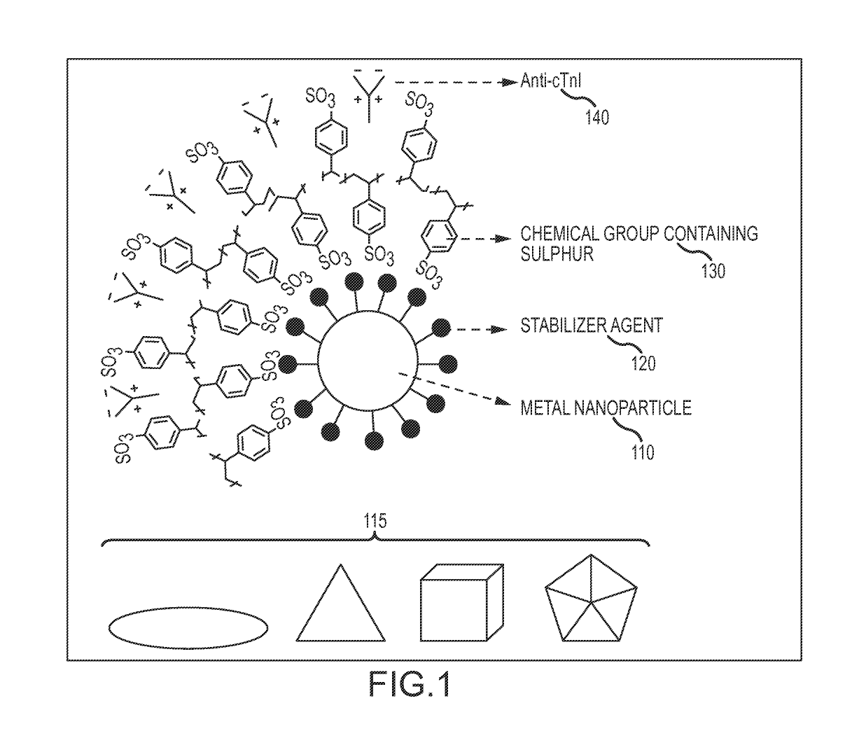

The shape of metallic nanoparticles plays a key role in determining their optical properties. In multiple embodiments of the present invention, the metallic nanoparticles are fabricated as a variety of shapes, including but not limited to spheres, pyramids, cubes, polyhedrons 115 and rods as can be seen in FIG. 1.

As previously described and explained below in further detail, an anti-cTnl antibody is conjugated to a metallic nanoparticle. FIG. 1 shows a high level depiction of the conjugation between anti-cTnl and a metallic nanoparticle. A metallic nanoparticle associated with a stabilizing agent 120 is combined with a chemical group containing sulphur. In one embodiment of the present invention the sulphur group is polystyrene sulfonate. Interposed between the sulphur group and the nanoparticle is a stabilizing agent 120. A stabilizer is a chemical which tends to inhibit the reaction between two or more other chemicals, in this case, the interaction between the metallic nanoparticle and the sulphur group. It can be thought of as the antonym to a catalyst. The chemical group thereafter is receptive to an anti-cTnl antibody 140.

As one of reasonable skill in the relevant art will appreciate, the size and shape of the nanoparticles can vary based on the desired detection protocol. Other metallic nanoparticle shapes such as cubes, polyhedrons, rods and spheres are contemplated and applicable to the scope of the present invention. While the present invention uses the structure of a nanorod as an exemplar of a conjugated anti-cTnl antibody, the use of other metallic nanoparticle shapes is possible. Similarly, the choice of PSS as a chemical group use to conjugate the anti-cTnl antibody to the nanoparticle may vary without departing from the scope of the present invention.

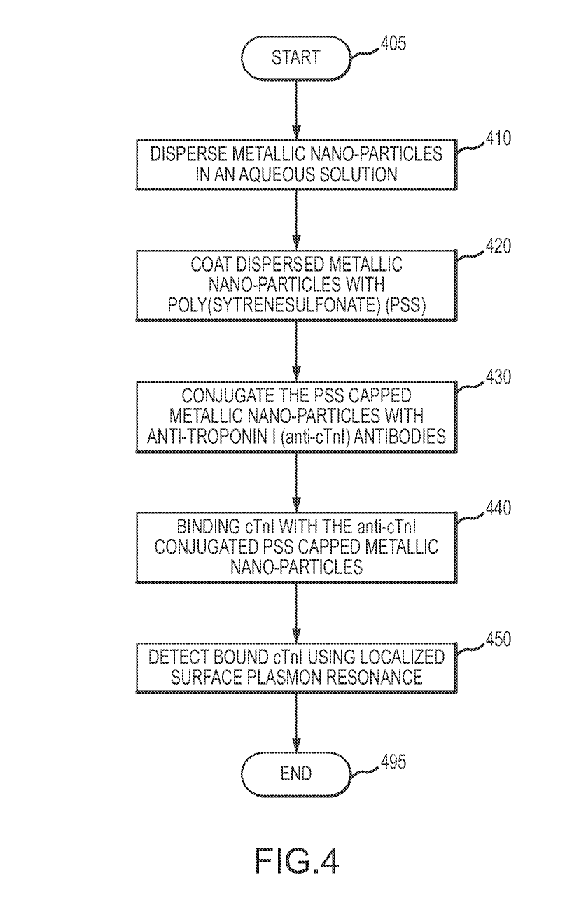

According to one embodiment of the present invention, a fast, simple, precise and low-cost methodology directly detects and quantifies the “gold standard” biomarker for acute myocardial damage, cTnl, in a small volume of whole blood by using metallic nanoparticles conjugated with anti-cTnl antibodies. FIG. 4 presents a flowchart of one methodology for the detection and quantification of cTnl in a blood sample according to the present invention. The process begins 405 with fabrication of monodisperse metallic nanoparticles (MNPs) 110 via a colloidal chemistry method 410 by using, in one embodiment, salts as metallic precursors. In one version of the present invention, a metal seed solution is fabricated by adding 0.6 ml of ice-cold solution of 10 mM NaBH4 to 10 mL of 0.25 mM HAuCl4 prepared in 0.1 M cetyltrimethylammonium bromide (CTAB, a stabilizer agent) 120 solution, under vigorous stirring for approximately 2 minutes. As one of ordinary skill in the art will recognize, the immediate transformation of the original yellow color to brown is indicative of the formation of gold seeds. In one embodiment of the present invention, the seeds were aged for approximately 8 hours in order to allow for the hydrolysis of unreacted NaBH4. The growth procedure described above can be scaled up to obtain a 100 mL or larger dispersion of the MNPs.

Thereafter, the MNPs are coated 420, according to one embodiment of the present invention, with a ligand such as polystyrene sulfonate (PSS), 130 having a molecular weight of 70,000 daltons via centrifuging 10 mLs of the MNP dispersion at 13,000 g/min for 20 minutes, discarding the supernatant and redispersing the precipitate in 5 ml of ultra-pure water.

Ligands are bound to the nanoparticle surface by some attractive interaction, examples including but not limited to chemisorption, electrostatic attraction and hydrophobic interaction, most commonly provided by a head group of the ligand molecule. Various chemical functional groups possess a certain affinity to inorganic surfaces, for example, thiol to gold. The choice of ligand molecules depends on such features as the material of the nanoparticle core, the particle size and the solvent and includes but is not limited to monothiol-terminated alkyl carboxylic acids, bidentate ligands, dendrons, polyethylene glycol (PEG) derivatives, polyvinylpyrrolidone (PVP), poly(ethylene oxide), oligomeric phosphines, silica shell encapsulation, biotin, avidin, peptides, carbohydrates, nucleic acids and polystyrene. While the coating of the nanoparticle can use a variety of molecules, the present invention is largely discussed using polystyrene sulfonate resulting in PSS-capped MPNs and the use of PSS should not be deemed as limiting as to the scope of the invention.

Continuing with the prior process, 1 mL of PSS (2.5 mg/L) is subsequently added drop-wise under vigorous stirring. After adding PSS solution, the mixture is kept undisturbed for 0.5 hour and then, according to one embodiment of the invention, the mixture is centrifuged twice at 12,500 g/min to remove any excess polyelectrolyte. The resultant PSS-capped MNPs are dispersed in 5 mL of PBS buffer (pH=7.0).

The process, according to one embodiment of the present invention, continues with the PSS-capped MNPs being conjugated 430 with anti-cTnl antibodies 140 as follows. The PSS-capped MNPs are mixed with excess amount of anti-cTnl antibody solution (50 μg/ml in PBS buffer, pH=7.0) for 30 minutes under stirring. The mixture is then centrifuged and the supernatant discarded in order to remove unbound antibodies. The PSS-capped MNPs conjugated with anti-cTnl antibodies are redispersed into PBS buffer. Thereafter, the PSS-capped MNPs conjugated with anti-cTnl antibodies are stored, in one embodiment of the present invention, at approximately 4 to 8 degrees Celsius for up to approximately one year.

The PSS-capped MNPs conjugated with anti-cTnl antibodies are stored at approximately 15 degrees Celsius for up to approximately 60 days. In yet another embodiment of the present invention, the PSS-capped MNPs conjugated with anti-cTnl antibodies are stored at approximately 18 to 50 degrees Celsius for up to approximate 14 days. The stability of the PSS-capped MNPs conjugated with anti-cTnl antibodies at ambient temperatures allows this biosensor to be stored in places void of refrigeration capabilities, thus further facilitating the convenience and portability of the methodology described herein.

The detection and quantification of cTnl is determined using the prepared PSS-capped MNPs conjugated with anti-cTnl antibodies. According to one embodiment of the present invention, a whole blood sample (approximately 0.5 ml) is mixed (1 minute, under vortex or similar mixing) with the PSS-capped MNPs conjugated with anti-cTnl antibodies (approximately 0.5 ml) (440), resulting in a total volume of approximately 1 ml. One skilled in the relevant art will appreciate that the volumes listed in this embodiment are exemplary only and that in other embodiments other volumes and quantities while maintaining their representative relationship may be used without departing from the scope of the present invention.

For example, in one embodiment of the present invention, the whole blood sample to be analyzed for cTnl is “fresh” (for example, analyzed approximately immediately after donation). In another embodiment of the present invention, the whole blood sample to be analyzed for cTnl has been stored for up to approximately 20 days. In yet other embodiments of the present invention serum and plasma derived from blood sample can combined with the ligand-capped MNPs conjugated with anti-cTnl antibodies for analysis.

cTnl has a high positive charge (pl 9.87) and, consequently, attracts negatively charged molecules such as heparin, which can interfere with the antibody-antigen interaction. Accordingly, in yet another embodiment of the present invention, the blood sample is void of heparin or similar blood thinning agents.

In another embodiment of the present invention, the blood sample is void of ethylennediaminetetraacetic acid (EDTA), as the presence of this anticoagulant results in cTnl analytical discrepancies because EDTA can cause partial unfolding of the calcium-dependent Troponin complex.

Once the sample has been mixed with the ligand-capped MNPs conjugated with anti-cTnl antibodies, the mixture is inserted into a disposable polymeric cuvette compatible with a spectrophotometer and run for, in one embodiment, approximately 1 minute 450. In this manner, using the ligand-capped MNPs conjugated with anti-cTnl antibodies of the present invention as described herein, cTnl is detected and quantified in approximately 4 minutes ending the process 495.

In one embodiment of the present invention, the percent coefficients of variation (% CV) obtained from whole blood sample measurements are less than or equal to approximately 10%.

The methodology presented herein, that is using ligand-capped MNPs conjugated with anti-cTnl antibodies employed to analyze cTnl concentrations in blood, can be used to identify patients presenting symptoms such as chest pain, nausea, headache, shortness of breath, general epigastric discomfort, sweating, heartburn and/or indigestion, arm pain and/or upper back pain may indeed be experiencing an AMI.

In addition to cTnl detection and quantification, the methodology described herein can be applied in a wide variety of detection and diagnostic tests. These tests include but are not limited to: qualitative/quantitative detection of proteins, nucleic acids (DNA and RNA), other molecules, hepatitis A, B and C, dengue fever and other pathological diagnoses. For detection, a small volume of sample is used (approximately 0.5 mL). In one embodiment of the present invention, a sample volume of 20 uL of whole blood is used. For sensing/detection tests in fluid samples such as urine, sweat, saliva and water, similar sample volumes are used. This analytical approach boasts a low-cost, fast response (four minutes or less, for each run), high sensitivity (detection range: 0.06 ng/ml up to 40 ng/ml) and easy execution (as the equipment needed to run the test consists of commercial UV/Vis or V is/NIR spectrophotometers that are routinely used in hospitals, laboratories and even in small medical offices that perform conventional biochemical tests) for detecting and quantifying cTnl and the like.

Moreover, the methodology described herein is suitable for monitoring heart conditions of cardiac and renal patients during therapeutic processes and surgical procedures that make use of anti-coagulant drugs. In addition, the methodology described herein is also suitable for monitoring patients that are utilizing drugs which interfere with circulation dynamics and related proteins.

According to one aspect of the present invention, the presence of cTnl is detected using Localized Surface Plasmon Resonance (LSPR). Recall that surface plasmon resonance is the collective oscillation of valence electrons in a solid stimulated by incident light. Resonance is established when the frequency of light-photons matches the natural frequency of surface electrons oscillating against the restoring force of positive nuclei. Surface plasmon resonance in nanometer-sized structures is called localized surface plasmon resonance. Accordingly, LSPR is, according to one embodiment of the present invention, the basis for measuring absorption of a material (in this case cTnl) onto the surface of metallic nanoparticles.

In the case of localized surface plasmon, light interacts with particles much smaller than the incident wavelength which leads to a plasmon that oscillates locally around the nanoparticle. As the local dielectric environment changes so too does a localized surface plasmon resonance. This change is typically seen as a shift in wavelength of the measured light.

According to one embodiment of the present invention, cTnl can be detected by using spectroscopy which measures this shift in wavelength. Spectroscopy is the study of the interaction between matter and radiated energy. Historically, spectroscopy originated through the study of visible light dispersed according to its wavelength. In such a technique, the absorption of energy due to the nanoparticles is measured by recording the wavelength dependence of the light passing through a sample. Because the shape and size of the metallic nanoparticle dictate the spectral signature of its plasmon resonance, the ability to change these two parameters is an important result of the present invention. In the present invention the adaptation of nanorods having an aspect ratio between 4.0 and 5.0 (optimally 4.5) provide an optimal refractive-index sensitivity. Nanorods show a higher sensitivity to LSPR than do any other nano-shaped particles including spheres and triangles.

LSPR sensing as a means by which to measure wavelength-shift of an LSPR extension curve is a function of changes in the local dielectric environment caused by analyte absorption. According to one embodiment of the present invention, the signal generated by the LSPR is detected by a spectrophotometer. In general, this instrument consists of three main components: a light source, the sample and a detector. In one version of the present invention, a LSPR signal is detected by a spectrophotometer and is indicative of the presence and concentration of cTnl in the sample. As can be appreciated by one of reasonable skill in the relevant art, due to the increasing sensitivity and portability of spectrophotometers (which can be found, for example, in hospitals, laboratories and ambulances), the methodology of the present invention can have wide applicability and impact.

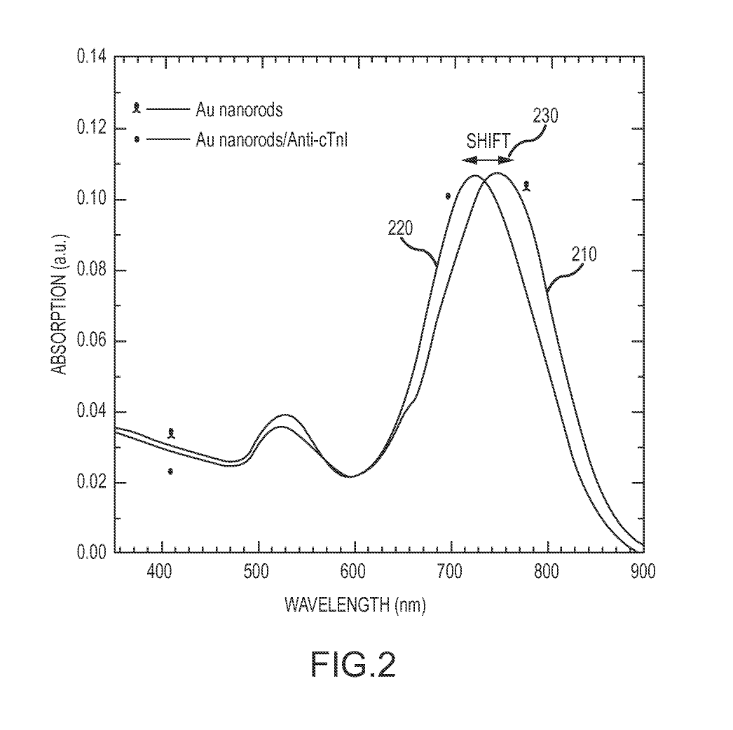

As a result of cTnl binding with anti-cTnl antibodies conjugated with PSS Metallic nanoparticles, the wavelength shift of absorption measured by LSPR in gold nanoparticles has been shown to approximate 20 to 50 nm. FIG. 2 shows a wavelength versus absorption plot of gold nanorods versus gold nanorods conjugated with anti-cTnl antibodies. As seen, the absorption of light by both the gold nanorods with anti-cTnl antibodies 220 and the gold nanorods void of anti-cTnl antibodies 210 are approximately 0.11 atomic units. However, those nanorods which are conjugated with anti-cTnl antibodies possess a different surface environment which decreases the observed wavelength absorption by approximately 50 nm 230.

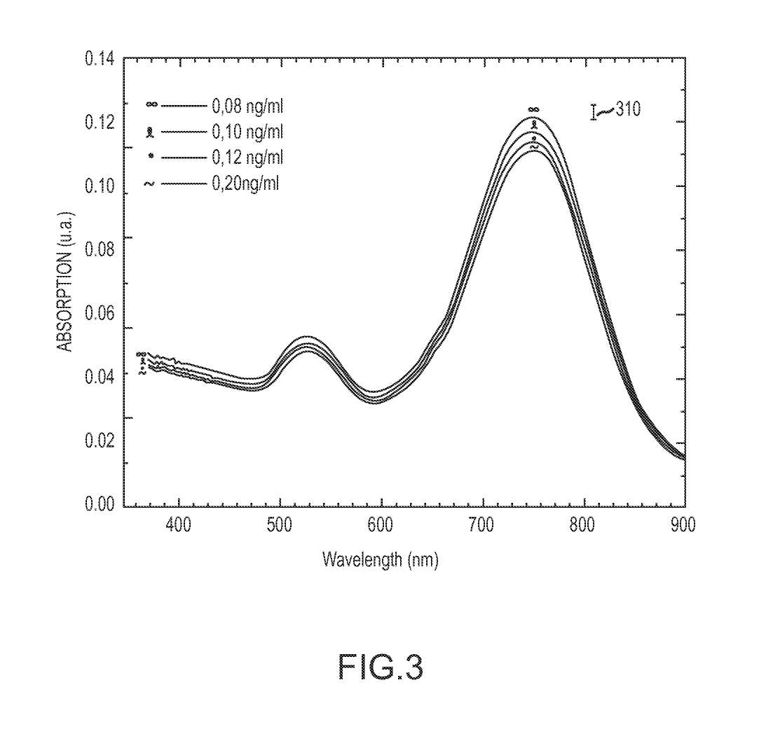

Referring in addition to FIG. 3, one skilled in the relevant art will see that the absorption of energy for gold nanorods bound with anti-cTnl antibodies conjugated with PSS capped MNPs varies according to the concentration of the bound material, that is cTnl bound to anti-cTnl antibodies conjugated to PSS capped MNPs. In the case illustrated in FIG. 3 the absorption of light varies 310 from approximately 0.105 atomic units to approximately 0.120 atomic units based on a concentration variance from 0.20 ng/ml to 0.08 ng/ml of bound anti-cTnl antibodies conjugated with PSS-capped gold nanorods. Accordingly, the present invention, using LSPR can detect not only the present of cardiac-specific Troponin (cTnl), but can also quantify its concentration.

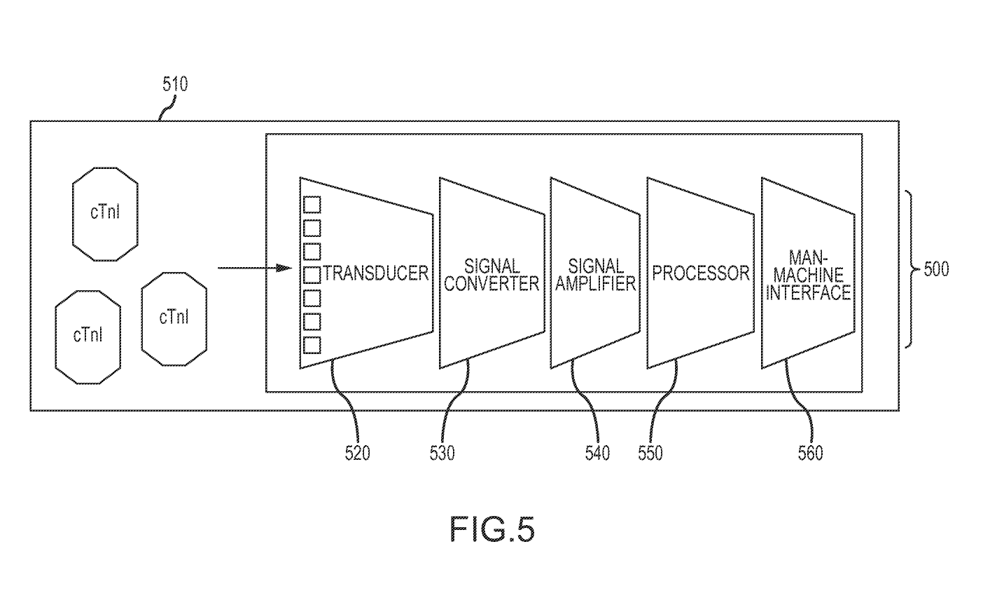

Another aspect of the present invention is the application of the detection of cTnl using anti-cTnl antibodies conjugated with PSS capped nanoparticles using a point of care device. According to one embodiment of the present invention, such a point of care device can identify and measure the concentration of cTnl using as little as 150 μL sample of blood (a finger tip drop of blood) in under 4 minutes. FIG. 5 depicts a high level schematic of a modified metallic nanoparticle conjugated to anti-cTnl antibodies. In this example, a sample of patient blood is introduced to a pre-prepared vile of anti-cTnl antibodies conjugated with PSS capped metallic nanoparticles. According to one embodiment of the present invention, the metallic nanoparticles are gold nanorods having an aspect ratio between 4.0-5.0 with an optimal aspect ratio of 4.5. The prepared sample holder can, in one embodiment of the present invention, be a polystyrene microcuvette prepared to include anti-cTnl antibodies conjugated to PSS capped gold nanorods. In other embodiments of the present invention, the microcuvette can be prepared with other conjugated antibodies specific for diagnostic tests of other diseases such as Hepatitis, Diabetes, and other antigen specific diseases in which an antigen can bind with a conjugated antibody. Moreover, while the present invention has been described in terms of a blood sample, the means of detection described herein can be equally applied to other fluid samples such as urine, saliva or water or the components of blood such as serum or plasma. In each application described above, the microcuvette can be specifically adapted to possess specific antibodies that will bind with an antigen present in a sample and thereafter detected using LSPR.

With reference to FIG. 5, and continuing with the exemplar of detection of cTnl, a patient blood sample is introduced to a microcuvette pre-charged with an anti-cTnl antibody conjugated with PSS capped gold nanorods. As the blood flows into the microcuvette by capillarity, any cTnl antigen will quickly bind with anti-cTnl antibodies. As the cTnl antibodies are conjugated with PSS capped gold nanorods, the now bound cTnl antigens are associated with the conjugated PSS capped gold nanorods.

The presence of cTnl bound to anti-cTnl antibodies conjugated with PSS capped gold nanorods is detected by spectroscopy and specifically LSPR. As shown by the point of care device 500 shown in FIG. 5, PSS capped gold nanorods conjugated with anti-cTnl antibodies act as transducers 520 that when combined with the sample 510 and excited by light of a specific wavelength produce a measurable plasmon resonance that can be converted into an electrical signal.

Anti-cTnl antibodies conjugated with PSS-capped gold nanorods possess a characteristic plasmon resonance when they are unbound. Upon binding with cTnl, the wavelength of light absorbed by the anti-cTnl antibodies conjugated with PSS-capped gold nanorods shifts. This shift in wavelength conclusively identifies the presence of cTnl. Moreover, the degree of absorption shift from a known baseline of unbound anti-cTnl antibodies conjugated with PSS-capped gold nanorods can be used to quantify the concentration of cardiac-specific Troponin I in the sample.

Using LSPR, a signal can be created based on the shift in absorption wavelength. This signal, which is analog in nature, can be converted to a digital signal using a standard analog-to-digital converter 530 as would be known to one of reasonable skill in the relevant art. The signal can thereafter be amplified 540 and used as a basis of input for processor 550 so as to produce an interpretable value for display on a user interface 560 or further processing. Plasmon resonance of the gold nanorods produces, through spectrometry, a measurable signal. In this regard, the gold nanorods, or for that matter, any metallic nanoparticle, is a transducer, acting to convert light energy into mechanical energy (resonance) that can be measured as an electrical signal by virtual of a wavelength/absorption shift. The signal represents one or more observed intensities and wavelengths of light. Once converted and amplified, the signal can be interpreted based on known parameters.

In the example of anti-cTnl antibodies conjugated with PSS capped gold nanorods, a known degree of light absorption at a certain wavelength can be expected and programmed into a processor. This value can then be used as a point of comparison to data determined by plasmon resonance of anti-cTnl antibodies conjugated with PSS capped gold nanorods bound with cTnl. In this example, a decreased absorption wavelength from the baseline value conclusively detects the presence of cTnl with the degree of absorption at that wavelength being indicative of the concentration of cTnl.

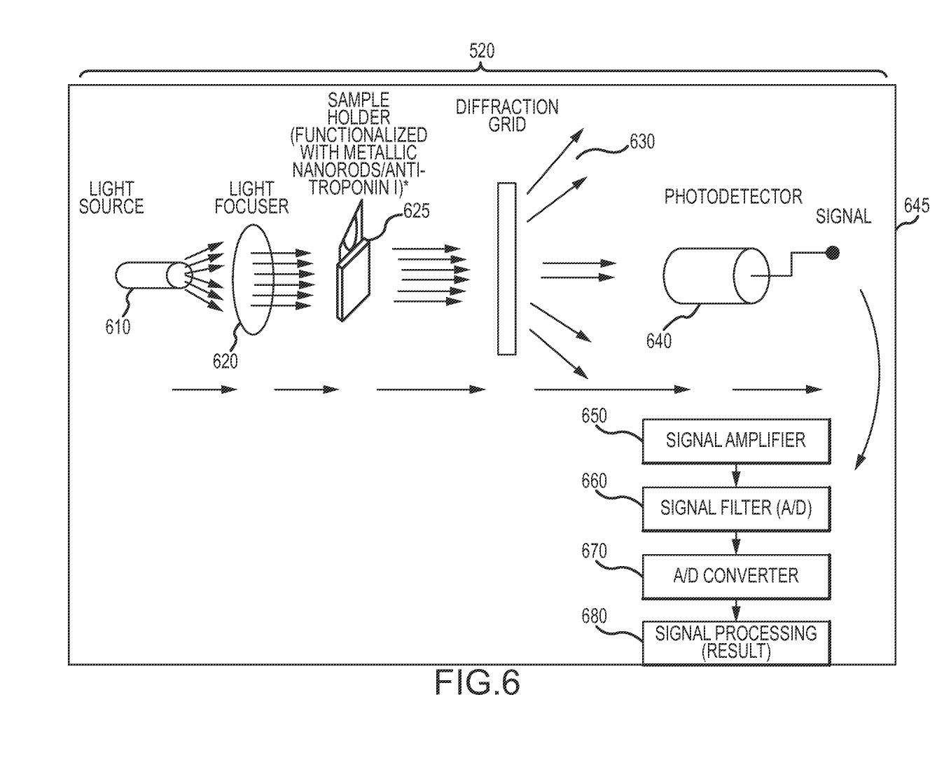

FIG. 6 is a high level functional block diagram of a point of care device using LSPR and anti-cTnl antibodies conjugated to PSS capped metallic nanoparticles to detect cTnl in patent blood sample, according to one embodiment of the present invention. A light source 610 is focused 620 and directed toward a sample holder 625 comprising anti-cTnl antibodies conjugated with PSS capped metallic nanoparticles and a patient blood sample. Light exiting the sample holder is, in one embodiment, passed through a diffraction grid 630 and received by a photodetector or photosensor 640 which produces an electrical signal. As one of reasonable skill in the art will appreciate the detection of a cTnl antigen in the sample of blood is based on a shift in the localized surface plasmon resonance of gold nanorods produced by the introduction of light of a specific wavelength. The degree of absorption and wavelength of the light vary according to the binding effect of a cTnl antigen to an anti-cTnl antibody conjugated with PSS capped metallic nanoparticles.

The signal 645 is amplified by a signal amplifier 650 and then subject to one or more analog filters 660. Thereafter the analog input is converted to a digital signal by an analog-to-digital converter before it arrives at processor 650. The processor 650 thereafter manipulates and interprets the signal based on known and observed parameters to convey a result of the analysis in a user friendly manner. According to one embodiment of the present invention, the point of care device converts the observed absorption and wavelength shift into a measurable detection of cTnl.

As one of reasonable skill in the relevant art will appreciate, a wide variety of specific components can be utilized to create and detect an excited state of PSS capped metallic nanorods conjugated to anti cTnl antibodies bound to cTnl antigens. While the above description generally describes a functional schematic of a point of care device capable of introducing a sample holder comprising anti-cTnl antibodies conjugated with PSS capped metallic nanoparticles and a patient blood sample to determine the presence of cTnl antigens using LSPR, one skilled in the at will recognize that the specific aspects of each of the depicted components, and of those omitted for sake of brevity, may take many forms. Indeed, many of the functional components depicted in FIG. 6 may be performed by a single device or the functionality of a specific depicted component may be accomplished by multiple devices. The specific choice of such components does not diminish the contemplated scope of the present disclosure, that is the detection of a cTnl antigen in a patient blood sample using a conjugation of anti-cTnl antibodies and PSS capped metallic nanoparticles and LSPR.

Although the invention has been described and illustrated with a certain degree of particularity, it is understood that the present disclosure has been made only by way of example and that numerous changes in the combination and arrangement of parts can be resorted to by those skilled in the art without departing from the spirit and scope of the invention. Likewise, the particular naming and division of the modules, managers, functions, systems, engines, layers, features, attributes, methodologies, and other aspects are not mandatory or significant, and the mechanisms that implement the invention or its features may have different names, divisions, and/or formats. Furthermore, as will be apparent to one of ordinary skill in the relevant art, the modules, managers, functions, systems, engines, layers, features, attributes, methodologies, and other aspects of the invention can be implemented as software, hardware, firmware, or any combination of the three. Of course, wherever a component of the present invention is implemented as software, the component can be implemented as a script, as a standalone program, as part of a larger program, as a plurality of separate scripts and/or programs, as a statically or dynamically linked library, as a kernel loadable module, as a device driver, and/or in every and any other way known now or in the future to those of skill in the art of computer programming. Additionally, the present invention is in no way limited to implementation in any specific, device, programming language, or for any specific operating system or environment. Accordingly, the disclosure of the present invention is intended to be illustrative, but not limiting, of the scope of the invention, which is set forth in the following claims.

Although claims have been formulated in this application to particular combinations of features, it should be understood that the scope of the disclosure herein also includes any novel feature or any novel combination of features disclosed either explicitly or implicitly or any generalization or modification thereof which would be apparent to persons skilled in the relevant art, whether or not such relates to the same invention as presently claimed in any claim and whether or not it mitigates any or all of the same technical problems as confronted by the present invention. The Applicant hereby reserves the right to formulate new claims to such features and/or combinations of such features during the prosecution of the present application or of any further application derived there from.

1. A method for detecting cardiac-specific Troponin I (cTnI), the method comprising:
dispersing metallic nanoparticles in an aqueous Cetyl Trimethyl Ammonium Bromide (CTAB) solution forming a plurality of dispersed metallic nanoparticles;
coating the plurality of metallic nanoparticles with a ligand forming a plurality of ligand-capped metallic nanoparticles wherein a first dispersion containing the plurality of ligand-capped metallic nanoparticles has a pH of substantially 7.0;
conjugating the plurality of ligand-capped metallic nanoparticles with anti-Troponin I (anti-cTnI) antibodies;
mixing the anti-cTnI antibodies conjugated with ligand-capped metallic nanoparticles with a patient sample wherein the cTnI within the sample binds with the anti-cTnI antibodies conjugated with ligand-capped metallic nanoparticles wherein a second dispersion containing the plurality of anti-cTnI antibodies conjugated with ligand-capped metallic nanoparticles has a pH of substantially 7.0; and
detecting a presence of cTnI bound to the anti-TnI antibodies conjugated with ligand-capped metallic nanoparticles using a signal generated by Localized Surface Plasmon Resonance (LSPR).

2. The method for detecting cardiac-specific Troponin I according to claim 1, wherein anti-cTnI antibodies conjugated with ligand-capped metallic nanoparticles bound with cTnI shifts the signal generated by Localized Surface Plasmon Resonance relative to the signal generated by unbound anti-TnI antibodies conjugated with ligand-capped metallic nanoparticles.

3. The method for detecting cardiac-specific Troponin I according to claim 1, wherein the presence of cTnI bound to the anti-TnI antibodies conjugated with ligand-capped metallic nanoparticles can be detected at concentrations of cTnI from 0.002 ng/mL to and including 40 ng/mL.

4. The method for detecting cardiac-specific Troponin I according to claim 1, wherein the plurality of ligand-capped metallic nanoparticles and the plurality of anti-cTnI antibodies conjugated with ligand-capped metallic nanoparticles are dispersed in a Phosphate Buffered Saline (PBS) buffer.

5. The method for detecting cardiac-specific Troponin I according to claim 1, wherein the metallic nanoparticles are gold nanorods.

6. The method for detecting cardiac-specific Troponin I according to claim 5, wherein the gold nanorods have an aspect ratio from 4.0 to 5.0.

7. The method for detecting cardiac-specific Troponin I according to claim 5, wherein the gold nanorods have an aspect ratio greater or equal to 4.5.

8. The method for detecting cardiac-specific Troponin I according to claim 1, wherein the metallic nanoparticles are nanorods.

9. The method for detecting cardiac-specific Troponin I according to claim 1, wherein the metallic nanoparticles are gold nanoparticles.

10. The method for detecting cardiac-specific Troponin I according to claim 1, wherein the patient sample is whole blood.

11. The method for detecting cardiac-specific Troponin I according to claim 1, wherein the patient sample is serum.

12. The method for detecting cardiac-specific Troponin I according to claim 1, wherein the patient sample is plasma.

13. The method for detecting cardiac-specific Troponin I according to claim 1, wherein polystyrene sulfonate (PSS) has a molecular weight of 70,000.

14. The method for detecting cardiac-specific Troponin I according to claim 1, wherein the metallic nanoparticle dispersion is void of hydrochloric acid (HCl).

15. The method for detecting cardiac-specific Troponin I according to claim 1, wherein the ligand is polystyrene sulfonate (PSS).

16. A method for detecting cardiac-specific Troponin I, the method comprising:
conjugating a ligand-capped metallic nanoparticles with anti-Troponin I (anti-cTnI) antibodies;
binding one or more anti-cTnI antibodies conjugated with ligand-capped metallic nanoparticles with Troponin (cTnI) from a patient blood sample forming a detection sample; and
responsive to binding one or more anti-cTnI antibodies conjugated ligand-capped metallic nanoparticles with cTnI, detecting in the detection sample a spectral absorption shift in Localized Surface Plasmon Resonance (LSPR) of the metallic nanoparticles confirming a presence of cTnI.

17. The method for detecting cardiac-specific Troponin I according to claim 16, wherein metallic nanoparticles are gold nanoparticles.

18. The method for detecting cardiac-specific Troponin I according to claim 17, wherein the gold nanoparticles are gold nanorods.

19. The method for detecting cardiac-specific Troponin I according to claim 16, further comprising forming a plurality of ligand-capped metallic nanoparticles.

20. The method for detecting cardiac-specific Troponin I according to claim 16, wherein forming includes maintaining a pH of 7.0.

21. The method for detecting cardiac-specific Troponin I according to claim 16, further comprising forming a dispersion of anti-cTnI antibodies conjugated with ligand-capped metallic nanoparticles having a pH of substantially 7.0.

22. The method for detecting cardiac-specific Troponin I according to claim 16, wherein the a ligand-capped metallic nanoparticles are polystyrene sulfonate (PSS)-capped metallic nanoparticles.

23. A biosensor comprising cardiac-specific anti-Troponin I (anti-cTnI) antibodies conjugated with polystyrene sulfonate (PSS) capped metallic nanoparticles (MNP).

24. The biosensor of claim 23, wherein the pH of a dispersion containing the anti-cTnI antibodies conjugated with ligand-capped MNP antibody is substantially 7.0.

25. The biosensor of claim 23, wherein the metallic nanoparticles are a nanorods.

26. The biosensor of claim 23, wherein the nanorods have an aspect ratio between 4.0 and 5.0.

27. A system for detecting cardiac-specific Troponin I (cTnI), comprising:
a patient sample containing cTnI;
a plurality of anti-Troponin I (anti-cTnI) antibodies conjugated with ligand-capped metallic nanoparticles (MNP); and
a spectrophotometer operable to detect a shift of wavelength in light resulting from binding of cTnI to the anti-cTnI antibodies conjugated with ligand-capped metallic nanoparticles.

28. The system for detecting cTnI according to claim 27 wherein the spectrophotometer detects a signal generated by Localized Surface Plasmon Resonance (LSPR).

29. The system for detecting cTnI according to claim 27 wherein the metallic nanoparticles are gold nanorods.

30. The system for detecting cTnI according to claim 29, wherein the gold nanorods have an aspect ratio from 4.0 to 5.0.

31. The system for detecting cTnI according to claim 27, wherein the plurality of anti-cTnI antibodies conjugated with ligand-capped MNPs are polystyrene sulfonate (PSS)-capped MNPs.
